(19) United States
(12) Patent Application Publication
(10) Pub. No.: US 2017/0127955 A1
(43) Pub. Date: May 11, 2017

(54) MEASUREMENT APPARATUS AND MEASUREMENT METHOD

(71) Applicant: KYOCERA Corporation, Kyoto (JP)

(72) Inventors: Shotaro SUGITA, Kawasaki-shi, Kanagawa (JP); Takaaki WADA, Yokohama-shi, Kanagawa (JP)

(73) Assignee: KYOCERA Corporation, Kyoto (JP)

(21) Appl. No.: 15/322,615

(22) PCT Filed: Jul. 28, 2015
(86) PCT No.: PCT/JP2015/003773

§ 371 Date: Dec. 28, 2016

(30) Foreign Application Priority Data
Jul. 29, 2014 (JP) 2014-154210

(51) Int. Cl. A61B 5/026; A61B 5/00

(52) U.S. Cl. CPC A61B 5/0261; A61B 5/742; A61B 5/6843; A61B 5/6826; A61B 5/7405

(57) ABSTRACT

A measurement apparatus includes a light source that emits a measuring beam, a light receiver that receives scattered light of the measuring beam from a test site, a biological information generator that generates biological information based on output of the light receiver, and a controller. The controller permits emission of the measuring beam from the light source during a predetermined time slot.

### CROSS-REFERENCE TO RELATED APPLICATION

This application claims priority to and the benefit of Japanese Patent Application No. 2014-154210 filed Jul. 29, 2014, the entire contents of which are incorporated herein by reference.

### TECHNICAL FIELD

This disclosure relates to a measurement apparatus and a measurement method.

### BACKGROUND

An example of an existing measurement apparatus measures biological information by acquiring biological output information from a test site, such as a fingertip of a subject (user).

### SUMMARY

A measurement apparatus according to this disclosure includes:

a light source configured to emit a measuring beam;

a light receiver configured to receive scattered light of the measuring beam from a test site;

a biological information generator configured to generate biological information based on output of the light receiver; and

a controller;

such that the controller permits emission of the measuring beam from the light source during a predetermined time slot.

This disclosure may also be implemented as methods substantially corresponding to the above-described measurement apparatuses, and such methods are to be understood as included in the scope of this disclosure.

For example, a measurement method includes:

judging whether a current time is included in a predetermined time slot;

emitting a measuring beam onto a test site in contact with a contact interface when the current time is included in the predetermined time slot;

receiving scattered light of the measuring beam from the test site; and

generating biological information based on the scattered light.

### BRIEF DESCRIPTION OF THE DRAWINGS

In the accompanying drawings:

FIG. 1 is a functional block diagram schematically illustrating the structure of a measurement apparatus according to one of the embodiments of this disclosure;

FIG. 2 illustrates a subject holding the measurement apparatus;

FIG. 3 illustrates the relationship between contact pressure and emission of laser light; and

FIG. 4 is a flowchart illustrating an example of emission control of laser light by the controller.

### DETAILED DESCRIPTION

For example, an existing blood flow measurement apparatus that measures blood flow as the biological information irradiates a fingertip with laser light and measures the blood flow based on scattered light from the blood flow in a capillary at the fingertip.

When the subject measures biological information using a dedicated biological information measurement apparatus, however, the subject might forget to measure the biological information, or the subject might be unable to measure the biological information if the place or circumstances do not permit use of the biological information measurement apparatus. In this way, a conventional biological information measurement apparatus is not always convenient, and in some cases it may be difficult for a subject to measure and record biological information continuously on a regular basis.

It would therefore be helpful to provide a measurement apparatus and measurement method that are more convenient.

The following describes one of the disclosed embodiments in detail with reference to the drawings.

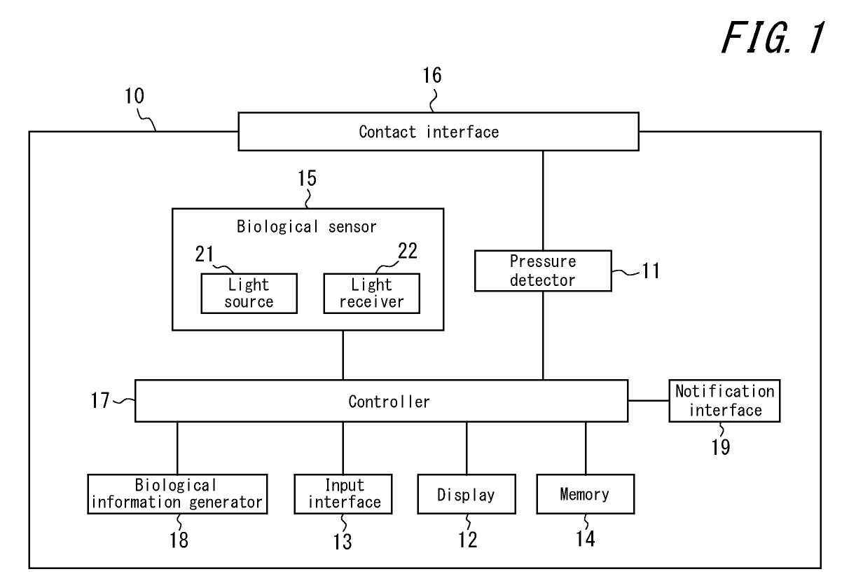

FIG. 1 is a block diagram schematically illustrating the structure of a measurement apparatus according to one of the disclosed embodiments. The measurement apparatus 10 may, for example, be configured by a mobile phone. As illustrated in FIG. 1, the measurement apparatus 10 includes a pressure detector 11, a display 12, an input interface 13, a memory 14, a biological sensor 15, a contact interface 16, a controller 17, a biological information generator 18, and a notification interface 19.

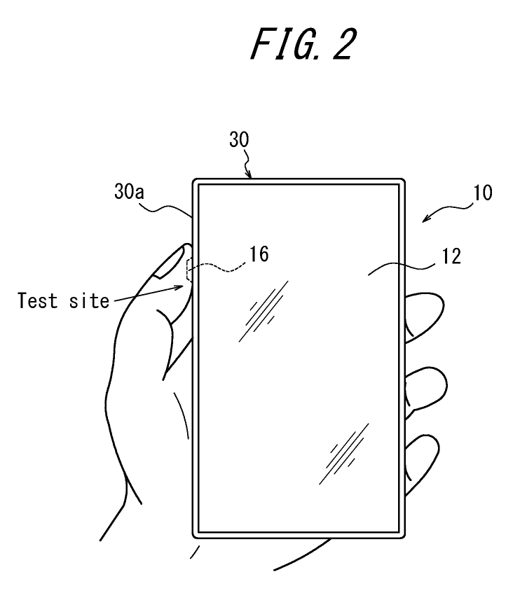

The measurement apparatus 10 measures biological information at a test site of the subject holding the measurement apparatus 10 while the test site is in contact with the contact interface 16. FIG. 2 illustrates the subject holding the measurement apparatus 10 in the left hand. As illustrated in FIG. 2, the contact interface 16 is disposed on one side 30a of a body 30 of a mobile phone, which is the measurement apparatus 10. The contact interface 16 may be disposed at a position that is naturally contacted by the test site when the subject holds the measurement apparatus 10. The biological information measured by the measurement apparatus 10 may be any biological information that can be measured using the biological sensor 15. In this embodiment, as one example, the measurement apparatus 10 is described as measuring the subject's amount of blood flow, which is information related to blood flow.

In FIG. 1, the pressure detector 11 is an example of a contact state detector that detects the contact state of the test site on the contact interface 16. As the contact state, the pressure detector 11 detects the contact pressure of the test site on the contact interface 16. The pressure detector 11 may, for example, be configured by a piezoelectric element. The pressure detector 11 is connected to the controller 17 and transmits the detected contact pressure to the controller 17 as a pressure signal. Accordingly, when the test site is in contact with the contact interface 16, the pressure detector 11 detects the contact pressure, from the test site, acting on the contact interface 16 and transmits the detected contact pressure to the controller 17 as a pressure signal.

The display 12 is a display device configured by a well-known display such as a liquid crystal display, an organic EL display, an inorganic EL display, or the like. For example, under control by the controller 17, the display 12 displays the measured biological information.

The input interface 13 accepts operation input from the user and may be configured, for example, using operation buttons (operation keys). The input interface 13 may be configured by a touchscreen, an input region that accepts operation input from the user may be displayed on a portion of the display device that is the display 12, and touch operation input by the user to this input region may be accepted.

The memory 14 may be configured with a semiconductor memory, a magnetic memory, or the like. The memory 14 stores a variety of information, programs for causing the measurement apparatus 10 to operate, and the like and also functions as a working memory.

The memory 14 for example stores past biological information measured by the measurement apparatus 10. The memory 14 also, for example, stores the below-described usage history information.

The biological sensor 15 acquires biological measurement output from the test site. When the measurement apparatus 10 measures the amount of blood flow, as in this embodiment, then the biological sensor 15 includes a light source 21 and a light receiver 22.

The light source 21 emits laser light based on control by the controller 17. The light source 21 may, for example, be configured to irradiate the test site with laser light, as a measuring beam, that has a wavelength capable of detecting a predetermined component included in blood. An example of such a light source is a Laser Diode (LD).

The light receiver 22 receives scattered light of the measuring beam from the test site as biological measurement output. The light receiver 22 may, for example, be configured by a photodiode (PD). The biological sensor 15 transmits a photoelectric conversion signal of the scattered light received by the light receiver 22 to the controller 17.

The contact interface 16 is a portion that contacts the test site, such as a finger, in order for the subject to measure biological information. The contact interface 16 is, for example, configured by a plate-shaped member. The contact interface 16 is configured by a member that is transparent at least with respect to the measuring beam from the light source 21 and the scattered light from the test site.

The controller 17 is a processor that, starting with the functional blocks of the measurement apparatus 10, controls and manages the measurement apparatus 10 overall. The controller 17 is configured using a processor such as a Central Processing Unit (CPU) that executes a program prescribing control procedures. Such a program may, for example, be stored in the memory 14, in an external storage medium, or the like.

The controller 17 stores the usage history information in the memory 14. The usage history information is information related to the time slots when the subject uses the measurement apparatus 10. In this embodiment, the controller 17 judges that the subject is using the measurement apparatus 10 when, for example, an image or the like is displayed on the display 12. The controller 17 may judge that the subject is using the measurement apparatus 10 when, for example, input is provided from the input interface 13 based on an operation by the subject. In other words, upon judging that the subject is using the measurement apparatus 10, the controller 17 stores the time slot during which the measurement apparatus 10 is being used as usage history information in the memory 14. Each time that the controller 17 judges that the subject is using the measurement apparatus 10, the controller 17 updates the usage history information stored in the memory 14. The controller 17 may, for example, judge that the subject is using the measurement apparatus 10 when the backlight of the display 12 is ON, when a predetermined application is running, or the like.

When the contact pressure detected by the pressure detector 11 is within a predetermined pressure range, the controller 17 emits a measuring beam from the light source 21. By emitting a measuring beam from the light source 21, the controller 17 starts to acquire the biological measurement output in the biological sensor 15.

The predetermined pressure range can be any pressure range over which the pressure acting on the contact interface 16 from the test site allows measurement of the amount of blood flow. In particular, a pressure range over which the pressure acting on the contact interface 16 from the test site is suitable for measurement of the amount of blood flow may be selected. The pressure range suitable for measurement of the amount of blood flow may, for example, be a pressure range such that, based on the statistical relationship between pressure and measurement error, the error in the measurement result of the amount of blood flow falls within a predetermined error range.

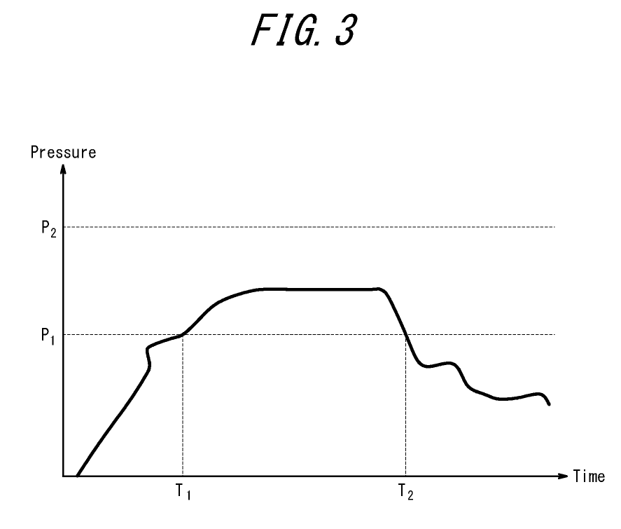

FIG. 3 illustrates the relationship between contact pressure and emission of laser light. In FIG. 3, the vertical axis represents strength of contact pressure, and the horizontal axis represents elapsed time. P₁ and P₂ are respectively the lower limit and upper limit of a predetermined pressure range. In other words, the predetermined pressure range is the range between P₁ and P₂. When the contact pressure is P₁ or greater and less than P₂ (T₁ in FIG. 3), the controller 17 emits laser light from the light source 21. While emitting laser light from the light source 21, the controller 17 acquires biological measurement output from the biological sensor 15. When the contact pressure becomes less than P₁ (T₂ in FIG. 3), or when the contact pressure exceeds P₂, the controller 17 suspends emission of laser light from the light source 21.

The controller 17 of the measurement apparatus 10 according to this embodiment performs control to allow emission of a measuring beam from the light source 21 during at least one predetermined time slot. The predetermined time slot is determined by the controller 17 for example based on the usage history information stored in the memory 14. Based on the usage history information, the controller 17 for example determines the predetermined time slot to be a time slot during which the subject is highly likely to be using the mobile phone that is the measurement apparatus 10. For example, when the controller 17 refers to the usage history information and judges that the frequency of usage of the mobile phone by the subject is highest during the two hours from 19:00 to 21:00, the controller 17 can determine that the two hours from 19:00 to 21:00 are the predetermined time slot. The measurement apparatus 10 according to this embodiment for example can reduce the power consumption of the pressure detector 11 by performing control to activate the pressure detector 11 only during the predetermined time slot that is determined as mentioned above.

A plurality of the predetermined time slots may be provided during one day. For example, suppose that the usage history information for the mobile phone of an office worker who works during the day indicates that the mobile phone is often used during the two hours from 7:00 to 9:00, which correspond to the time to go to work, the two hours from 11:00 to 13:00, which include a break from work, and the two hours from 19:00 to 21:00, which correspond to the time to return home. In this case, based on the usage history information, the controller 17 may determine that the three time slots per day during which the subject is highly likely to be using the mobile phone, such as the two hours from 7:00 to 9:00, the two hours from 11:00 to 13:00, and the two hours from 19:00 to 21:00, are predetermined time slots. The number of time slots determined by the controller 17 is not limited to being three per day and may be any number, one or greater, of time slots per day.

The controller 17 can judge whether the current time is included in the predetermined time slot by, for example, a clock function such as a Real-Time Clock (RTC).

While emitting laser light from the light source 21, the controller 17 provides notification via the notification interface 19 of information related to emission of laser light. As the information related to emission of laser light, the controller 17 for example provides notification via the notification interface 19 of the fact that laser light is being emitted.

The controller 17 stores the biological information generated by the biological information generator 18 in the memory 14. After the controller 17 stores the biological information in the memory 14 once during the predetermined time slot, biological measurement output need not be performed again during the time slot in which the biological information was recorded. In other words, the controller 17 may perform control so as to acquire valid biological measurement output, from which biological information can be generated, only once per predetermined time slot.

Based on output of the light receiver 22 (biological information output), the biological information generator 18 generates biological information. The biological information generator 18 may be configured in the measurement apparatus 10 as an independent functional component that differs from the controller 17, as illustrated in FIG. 1, or may be configured as a portion of the controller 17.

Here, a technique for measurement by the biological information generator 18 of the amount of blood flow using the Doppler shift is now described. When measuring the amount of blood flow, the controller 17 causes laser light to be irradiated from the light source 21 onto body tissue (the test site) and receives scattered light that is scattered from the body tissue with the light receiver 22. The biological information generator 18 then calculates the amount of blood flow based on output related to the scattered light that was received.

In the body tissue, scattered light that is scattered from moving blood cells undergoes a frequency shift (Doppler shift), due to the Doppler effect, proportional to the speed of travel of the blood cells within the blood. The biological information generator 18 detects the beat signal due to interference between scattered light from still tissue and the scattered light from moving blood cells. This beat signal represents strength as a function of time. The biological information generator 18 then turns the beat signal into a power spectrum that represents power as a function of frequency. In this power spectrum of the beat signal, the Doppler shift frequency is proportional to the speed of blood cells, and the power corresponds to the amount of blood cells. The biological information generator 18 calculates the amount of blood flow by multiplying the power spectrum of the beat signal by the frequency and integrating.

Based on control by the controller 17, the notification interface 19 provides notification of information related to emission of a measuring beam. The notification interface 19 provides notification for example by a visual method using an image, characters, light emission, or the like; an auditory method using audio or the like; or a combination of these methods. In the case of providing notification with a visual method, for example the display 12 can be used as the notification interface 19, and the notification interface 19 can provide notification by displaying images or characters on the display 12. The notification interface 19 can, for example, be configured by a light emitting element, such as a Light Emitting Diode (LED), and can provide notification by emitting light. In the case of providing notification with an auditory method, the notification interface 19 for example can be configured by a sound output device, such as a speaker, and can provide notification by outputting an alarm sound, audio guidance, or the like. Notification by the notification interface 19 is not limited to a visual or auditory method. Any method recognizable by the user may be adopted.

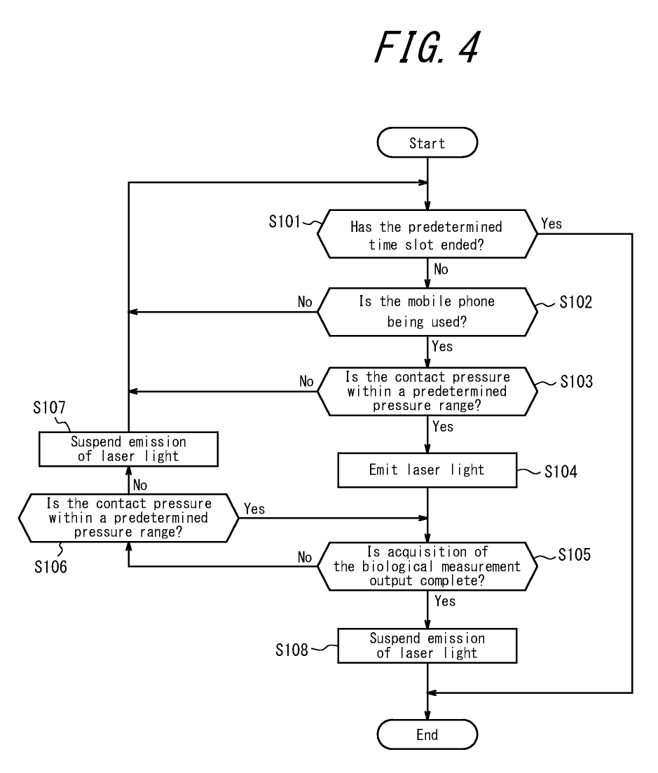

Next, an example of how the controller 17 performs emission control of laser light with respect to the light source 21 is described with reference to the flowchart in FIG. 4. The processing of the flowchart in FIG. 4 begins when the measurement apparatus 10 enters a state capable of measuring the amount of blood flow during a predetermined time slot. At the start of this processing, laser light is not being emitted from the light source 21.

The controller 17 judges whether the predetermined time slot has ended for example using the clock function of an RTC or the like (step S101).

When judging that the predetermined time slot has ended (step S101: Yes), the controller 17 ends the processing flow.

When judging that the predetermined time slot has not ended (step S101: No), i.e. when judging that the current time is included in the predetermined time slot, the controller 17 judges whether the mobile phone is being used, for example based on whether an image is displayed on the display 12 (step S102).

When the controller 17 judges that the mobile phone is not being used (step S102: No), this processing flow returns to step S101.

When judging that the mobile phone is being used (step S102: Yes), the controller 17 acquires information related to the contact pressure detected by the pressure detector 11 and judges whether the contact pressure on the contact interface 16 is within a predetermined pressure range (step S103).

When the controller 17 judges that the contact pressure on the contact interface 16 is not within a predetermined pressure range (step S103: No), this processing flow returns to step S101.

When judging that the contact pressure on the contact interface 16 is within a predetermined pressure range (step S103: Yes), the controller 17 emits laser light from the light source 21 (step S104). By emission of laser light, acquisition of biological measurement output by the biological sensor 15 begins.

Subsequently, the controller 17 judges whether the acquisition of the biological measurement output by the biological sensor 15 is complete (step S105).

When judging that acquisition of the biological measurement output is not complete (step S105: No), the controller 17 acquires information related to the contact pressure detected by the pressure detector 11 and judges whether the contact pressure on the contact interface 16 is within a predetermined pressure range (step S106).

When the controller 17 judges that the contact pressure is within a predetermined pressure range (step S106: Yes), the processing moves to step S105.

When judging that the contact pressure is not within a predetermined pressure range (step S106: No), the controller 17 suspends emission of laser light from the light source 21 (step S107). Processing then moves to step S101.

On the other hand, when judging that acquisition of biological measurement output is finished in step S105 (step S105: Yes), the controller 17 suspends emission of laser light from the light source 21 (step S108). Acquisition of the biological measurement output in the biological sensor 15 is completed in this way.

Once acquisition of the biological measurement output is complete, the biological information generator 18 generates biological information based on the biological measurement output. The generated biological information is stored in the memory 14 by the controller 17.

In this way, the measurement apparatus 10 according to this embodiment is implemented as a mobile phone and measures biological information by acquiring biological measurement output when contact pressure on the contact interface 16 is within a predetermined pressure range during a predetermined time slot. Therefore, the subject can easily measure biological information while using the mobile phone without going to any particular trouble to measure the biological information. The measurement apparatus 10 is thus more convenient.

Since the measurement apparatus 10 measures the biological information during a predetermined time slot, the measurement apparatus 10 can measure the biological information during the same time slot every day. By measuring the biological information once in each predetermined time slot, the measurement apparatus 10 does not measure the biological information again in the same predetermined time slot, thereby keeping power consumption down. Moreover, based on the usage history information, the measurement apparatus 10 determines a time slot during which the subject is highly likely to be using the mobile phone to be the predetermined time slot. Therefore, during each predetermined time slot, it is highly likely that biological information can be measured and stored.

Furthermore, the measurement apparatus 10 emits laser light from the light source 21 when the contact pressure is within a predetermined pressure range. Therefore, unnecessary power consumption while the biological sensor 15 is not acquiring biological measurement output can be suppressed. By the measurement apparatus 10 providing notification via the notification interface 19 of information related to emission of the measuring beam, the subject can recognize that biological measurement output is being acquired. Therefore, the subject can more easily make a conscious effort to keep the contact pressure constant during acquisition of the biological measurement output.

This disclosure is not limited to the above embodiment, and a variety of modifications and changes are possible. For example, the functions and the like included in the various components and steps may be reordered in any logically consistent way. Furthermore, components or steps may be combined into one or divided.

According to the above embodiment, the controller 17 has been described as determining the predetermined time slot based on the usage history information, but determination of the predetermined time slot is not limited to this method. For example, the predetermined time slot may be determined by the subject using the input interface 13 to input the predetermined time slot in advance.

The number of predetermined time slots may be determined by the controller 17 based on biological information generated in the past by the biological information generator 18. For example, the controller 17 may change the number of predetermined time slots per day.

For example, the number of predetermined time slots may be determined so as to increase based on biological information generated in the past by the biological information generator 18. The biological information generated in the past may be biological information generated during any time period, such as all of the biological information generated in the past, or the biological information generated during the previous week. The controller 17 for example may refer to the amount of blood flow as the biological information stored in the memory 14 during the previous week and judge that the blood flow has gotten worse. In this case, the controller 17 for example can increase the set number of predetermined time slots from three times per day to five times per day. Based on the usage history information, the controller 17 determines the two predetermined time slots that were added. For example, in addition to the times from 7:00 to 9:00, from 11:00 to 13:00, and from 19:00 to 21:00 provided as examples in the above embodiment, the controller 17 determines the times from 9:00 to 11:00 and 15:00 to 17:00 to be predetermined time slots. In this way, by increasing the number of predetermined time slots, more biological information can be gathered for the subject, allowing the subject's condition to be learned with higher accuracy. In some cases, the number of predetermined time slots may be set to decrease.

In the above embodiment, the measurement apparatus 10 has been described as being provided with the pressure detector 11, but the pressure detector 11 is not essential. Depending on the biological information that is measured, the measurement apparatus 10 may be provided not with the pressure detector 11 but rather with a contact state detector that detects the contact state on the contact interface 16, such as a contact detector that can judge whether the test site is in contact with the contact interface 16.

In the above embodiment, the measurement apparatus 10 has been described as measuring the amount of blood flow as biological information, but the biological information measured by the measurement apparatus 10 may be any other information instead. The measurement apparatus 10 may, for example, measure the subject's pulse, stress state, or the like, but these examples are not limiting. It is generally known that the stress state correlates with the degree of activation of the sympathetic nerve. When the measurement apparatus 10 measures the stress state, for example the controller 17 measures the stress state by estimating the degree of activation of the sympathetic nerve based on the interval between pulse beats acquired by the biological sensor.

As the information related to emission of laser light in the above embodiment, the controller 17 has been described as providing notification of the fact that laser light is being emitted, but the information related to emission of laser light is not limited to this example. As the information related to emission of laser light, the controller 17 may, for example, provide notification of the fact that laser light is going to be emitted before emission of laser light.

In the above embodiment, the case of the measurement apparatus 10 being implemented as a mobile phone has been described, but the form of the measurement apparatus 10 is not limited to a mobile phone. The measurement apparatus 10 may, for example, be implemented as the steering wheel of an automobile. When the measurement apparatus 10 is implemented as the steering wheel of an automobile, the contact interface 16 is, for example, disposed in a portion of the steering wheel at a position naturally contacted by the test site when the subject grips the steering wheel. As the usage history information, the memory 14 for example stores information related to the time slots during which the automobile engine is on. Based on the usage history information, the controller 17 for example determines the predetermined time slot to be a time slot during which the subject is highly likely to be using the automobile in which the measurement apparatus 10 is mounted. For example, when the subject using the automobile commutes to work in the automobile, the controller 17 refers to the usage history information to judge that the automobile is highly likely to be used during time slots corresponding to going to work and returning home. The controller 17 then determines the two hours from 7:00 to 9:00, which correspond to the time to go to work, and the two hours from 19:00 to 21:00, which correspond to the time to return home, to be the predetermined time slots. When the contact pressure detected by the pressure detector 11 is within a predetermined pressure range during the predetermined time slots determined in this way, the controller 17 acquires the biological measurement output by emitting laser light from the light source 21 and measures biological information.

As other examples, the measurement apparatus 10 may for example be implemented on any electronic device that is continuously held by the subject for a predetermined time (at least the time for acquiring biological measurement output), such as a mouse for operating a computer, a game controller, a tablet, and the like.

On devices that do not maintain a state of being held by the subject for a predetermined time, the measurement apparatus 10 can be implemented for example by adopting a configuration to provide notification via the notification interface 19, before emitting laser light, of the fact that laser light will be emitted to acquire biological measurement output. For example, the operation button of a washing machine may be used to implement the measurement apparatus 10. In this case, the contact interface 16 is disposed on the surface of the operation button. As the usage history information, the memory 14 for example stores information related to the time slot during which the operation button is operated. Based on the usage history information, the controller 17 for example determines the predetermined time slot to be a time slot during which the subject is highly likely to operate the operation button. When a predetermined operation is performed on the washing machine during the predetermined time slot determined in this way, the controller 17 provides notification via the notification interface 19 of the fact that laser light will be emitted to acquire biological measurement output. After recognizing the notification, the subject contacts a finger to the contact interface 16, and when the contact pressure detected by the pressure detector 11 enters a predetermined pressure range, the controller 17 acquires biological measurement output by emitting laser light from the light source 21, thereby measuring biological information. In this way, the subject can easily measure biological information without going to any particular trouble to measure the biological information, which is more convenient. As another example, the measurement apparatus 10 may be implemented as the operation button of a device that the subject uses on a daily basis, such as a vacuum cleaner, rice cooker, or the like.

The controller 17 may change the predetermined pressure range described in FIG. 3 in accordance with the strength of the subject's hold on the measurement apparatus 10. In this case, the controller 17 for example measures the strength with which the subject holds the measurement apparatus 10 in advance using the pressure detector 11. Without changing the extent of the pressure range (i.e. the interval between P₂ and P₁ in FIG. 3), the controller 17 may change the upper limit and lower limit of the predetermined pressure range. As long as acquisition of the biological measurement output is not affected, the controller 17 may determine that a range with smaller variation in pressure while the subject holds the measurement apparatus 10 is the predetermined pressure range.

In the above embodiment, the biological information generator 18 provided in the measurement apparatus 10 has been described as generating the biological information based on output of the light receiver 22, but the biological information is not limited to being generated by the biological information generator 18 provided in the measurement apparatus 10. For example, a server that is connected to the measurement apparatus 10 by a network that is wired, wireless, or a combination of both may be provided with a functional unit corresponding to the biological information generator 18, and the server that includes this functional unit may generate the biological information. In this case, the measurement apparatus 10 acquires the biological information output via the biological sensor 15 and transmits the acquired biological information output to the server via a separately provided communication interface. The server generates the biological information based on the biological information output and transmits the generated biological information to the measurement apparatus 10. The subject can view the biological information received by the measurement apparatus 10 by displaying the biological information on the display 12. When the server generates biological information in this way, the measurement apparatus 10 can, for example, be reduced in size as compared to when all of the functional units in FIG. 1 are implemented on one measurement apparatus 10.

1. A measurement apparatus comprising:
a light source configured to emit a measuring beam;
a light receiver configured to receive scattered light of the measuring beam from a test site;
a biological information generator configured to generate biological information based on output of the light receiver; and
a controller;
wherein the controller permits emission of the measuring beam from the light source during a predetermined time slot.

2. The measurement apparatus of claim 1, further comprising:
a contact state detector configured to detect a contact state of the test site on a contact interface;
wherein the controller causes the measuring beam to be emitted from the light source when judging that the contact state detected by the contact state detector is a predetermined contact state.

3. The measurement apparatus of claim 1, further comprising:
a memory configured to store a time slot of usage of the measurement apparatus by a subject;
wherein the controller determines the predetermined time slot based on usage history information stored in the memory.

4. The measurement apparatus of claim 3,
wherein the memory stores the biological information generated by the biological information generator in the past; and
wherein the controller further determines the predetermined time slot based on the biological information generated by the biological information generator in the past.

5. The measurement apparatus of claim 1, wherein the controller determines the predetermined time slot based on input to the measurement apparatus from a subject.

6. (canceled)

7. The measurement apparatus of claim 1, further comprising:
a notification interface;
wherein the controller provides notification via the notification interface of information related to emission of the measuring beam either before causing the measuring beam to be emitted from the light source or while causing the measuring beam to be emitted.

8. The measurement apparatus of claim 1, wherein the biological information includes information related to blood flow.

9. A measurement method comprising:
judging whether a current time is included in a predetermined time slot;
emitting a measuring beam onto a test site in contact with a contact interface when the current time is included in the predetermined time slot;
receiving scattered light of the measuring beam from the test site; and
generating biological information based on the scattered light.
